(19) United States
(12) Patent Application Publication
(10) Pub. No.: US 2007/0244426 A1
(43) Pub. Date: Oct. 18, 2007

(54) DUCK BILL SEPTUM COMBINATION

(75) Inventors: Charles C. Hart, Summerville, SC (US); John R. Brustad, Dana Point, CA (US); Jeremy J. Albrecht, Ladera Ranch, CA (US)

Correspondence Address: APPLIED MEDICAL RESOURCES CORPORATION, 22872 Avenida Empresa, Rancho Santa Margarita, CA, 92688, US

(73) Assignee: Applied Medical Resources Corporation, Rancho Santa Margarita, CA (US)

(21) Appl. No.: 11/279,600

(22) Filed: Apr. 13, 2006

(51) Int. Cl. A61M 1/00

(52) U.S. Cl. 604/30

(57) ABSTRACT

A duckbill check valve and septum combination includes a hollow elastomeric body having an open proximal end, a closed distal end and a pair of opposed flat portions that form a distal edge. The distal edge includes a slit having a periphery that is smaller than the periphery of the instrument inserted therethrough. The slit provides a zero seal in the absence of an instrument and an instrument seal in the presence of an instrument. A shield including a tubular member and a plurality of blades protruding distally from the distal end of the tubular member may be placed inside the check valve and operates to center and guide the surgical instrument through the blades before expanding the slit of the check valve. An instrument inserted into the check valve stretches the distal portion of the duckbill valve to form a septum-like seal around the inserted instrument.

### BACKGROUND

This invention generally relates to medical access devices and, more specifically, to a duckbill check valve and septum combination for use with trocars Mechanical trocars typically include a cannula defining a working channel and a seal housing which encloses valves that function to inhibit the escape of insufflation gasses. The seal housing may include a duckbill check valve and a septum seal. The duckbill seal serves to prevent the escape of pressurized gas through the cannula. The septum prevents pressurized gas from escaping when an instrument is within the fluid pathway and the duckbill valve is subsequently interrupted.

The cannula of the trocar is adapted to be positioned across the abdominal wall of a patient using an obturator that is initially inserted into the working channel of the cannula and then removed once the cannula is in place. Various elongated instruments may be inserted through the working channel of the cannula to reach and perform operative functions at a site within the abdomen. It is the function of the valves to engage the outer surface of such an instrument and form seals around the instrument to prevent the escape of insufflation gases.

The aforementioned arrangement of seals or valves may result in undesired friction, accumulated distance within the entire device, and a complication for manufacturing. Therefore, there is a need to simplify the seal portion of devices that may benefit from fewer parts and lighter construction.

### SUMMARY

The present invention is directed to sealing fluid passageways and, more specifically, to sealing fluid passageways with a single seal member that functions as a check valve and a septum seal. The seal member includes an elastomeric duckbill valve having a first, proximal, open end and support portion, and a second, distal end having an interruptible sealing portion. The distal sealing portion prevents retrograde gas flow as ambient pressure exerts a closing force upon opposing faces of the duckbill valve. An instrument inserted into the fluid passageway stretches the distal portion of the duckbill valve to form a septum-like seal around the inserted instrument.

In one embodiment, the invention includes a duckbill check valve for receiving an instrument therethrough. The duckbill check valve includes a substantially circumferential hollow elastomeric body having a first, proximal end and a second, distal end where the proximal end is open and the distal end is closed. The duckbill check valve also includes a pair of opposed substantially flat portions at an angle to a longitudinal axis of the elastomeric body. The substantially flat portions extend distally toward one another and intersect at the distal end of the elastomeric body, thereby forming an edge. The edge is substantially perpendicular to the longitudinal axis of the elastomeric body. The duckbill check valve also includes a slit along the edge at the distal end of the body. The periphery of the slit is no larger than the periphery of the instrument to be received by the check valve. The slit extends from an outer surface of the elastomeric body to an inner surface of the elastomeric body. The slit provides a zero seal in the absence of an instrument positioned within the slit and an instrument seal in the presence of an instrument positioned within the slit.

In one aspect, the elastomeric body has a substantially cylindrical shape. In another aspect, the elastomeric body has a substantially conical shape. In another aspect, the periphery of the slit is less the periphery of the instrument to be received by the check valve. In another aspect, the check valve also includes a reinforcing structural member at each end of the slit. Each of the structural members includes a protuberance projecting away from each of the flat portions of the check valve generally perpendicular to a plane formed by the longitudinal axis and the distal edge of the check valve and extending proximally into each of the flat portions of the check valve.

In another aspect, the check valve is formed of an elastomeric material including a low durometer polymer. In another aspect, the substantially flat portions are more elastic than the remainder of the elastomeric body. In another aspect, the substantially flat portions have a thickness smaller than the thickness of the remainder of the elastomeric body. In another aspect, the substantially flat portions have a lower durometer than the remainder of the elastomeric body. In another aspect, the check valve also includes a shield having a tubular member that has a proximal end and a distal end, and a plurality of blades protruding distally from the distal end of the tubular member. The shield is placed inside the check valve such that the blades engage the inner surface of the flat portions of the check valve. In another aspect, the shield is formed from a rigid plastic material. In another aspect, adjacent blades overlap one another. In another aspect, the shield operates to center and guide the surgical instrument through the blades before expanding the slit of the check valve.

In another embodiment, the invention includes a duckbill check valve for receiving an instrument therethrough. The duckbill check valve includes a substantially circumferential hollow elastomeric body having a first, proximal end that is open and a second, distal end that is closed. The check valve also includes a pair of opposed substantially flat portions at an angle to a longitudinal axis of the elastomeric body. The substantially flat portions extend distally and intersect at the distal end of the elastomeric body, thereby forming an edge. The edge is substantially perpendicular to the longitudinal axis of the elastomeric body. The flat portions include a plurality of bristles arranged in opposition. The bristles provide a zero seal in the absence of an instrument passing therethrough and an instrument seal in the presence of an instrument passing therethrough.

In one aspect, the elastomeric body includes a substantially cylindrical shape. In another aspect, the elastomeric body includes a substantially conical shape. In another aspect, the elastomeric body is formed of an elastomeric material including a low durometer polymer. In another aspect, the bristles include a substantially non-elastic material. In another aspect, the plurality of bristles includes a first, innermost layer of uncoated bristles and at least one outer layer of bristles coated with an elastomeric material. In another aspect, the check valve also includes a shield including, tubular member having a proximal end and a distal end, and a plurality of blades protruding distally from the distal end of the tubular member. The shield is placed inside the check valve such that the blades engage the inner surface of the flat portions of the check valve. In another aspect, the shield is formed from a rigid plastic material. In another aspect, adjacent blades overlap one another. In another aspect, the shield operates to center and guide the surgical instrument through the blades before separating the bristles.

These and other features of the invention will become more apparent with a discussion of the various embodiments in reference to the associated drawings.

### BRIEF DESCRIPTION OF THE DRAWINGS

FIG. 1 is a side elevation view of a prior art trocar partially cut away to illustrate an instrument seal valve;

FIG. 2 is a perspective view depicting a prior art trocar;

FIG. 3 is a perspective view depicting a prior art seal for a trocar;

FIG. 4 is a perspective view depicting a prior art duckbill valve having a cylindrical lumen and flat sealing portions in a closed condition;

FIG. 5 is a perspective view depicting the prior art duckbill valve of FIG. 4 in an interrupted, open condition;

FIG. 6 is a perspective view depicting a prior art duckbill valve having a tapered, conical lumen and flat sealing portions in a closed position;

FIG. 7 is a perspective view depicting the prior art duckbill valve of FIG. 6 in an interrupted, open condition;

FIG. 8 is a perspective view depicting a check valve of the present invention in a closed, sealed condition;

FIG. 9 is a perspective view depicting the check valve of FIG. 8 in an interrupted, sealed condition;

FIG. 10 is a side elevation view of a check valve of the present invention having a tapered, conical lumen and flat sealing portions in a closed position;

FIG. 11 is a section view taken from line 11-11 in FIG. 10;

FIG. 12 is a side elevation view of a check valve of the present invention having a cylindrical lumen and flat sealing portions in a closed position;

FIG. 13 is a section view taken from line 13-13 in FIG. 12;

FIG. 14 is a perspective view depicting a check valve of the present invention having reinforced portions;

FIG. 15 is a perspective view depicting a check valve of the present invention having fiber seal members in a closed, sealed condition;

FIG. 16 is a side elevation view of the check valve of FIG. 15;

FIG. 17 is a larger scale sectional view of a portion of the check valve of FIG. 15 taken from line 17 in FIG. 16;

FIG. 18 is a section view of a check valve of the invention having a shield inserted therein; and

FIG. 19 is a plan view, looking distally, of the shield of FIG. 18.

### DETAILED DESCRIPTION

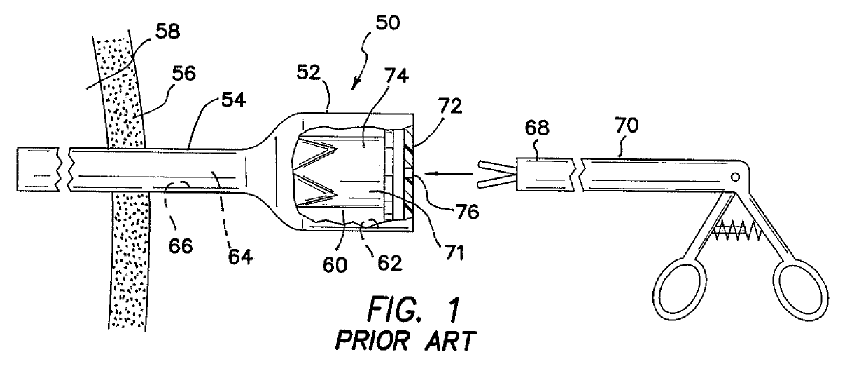

A trocar of the prior art is illustrated in FIGS. 1 and 2 and is designated generally by the reference numeral 50. The trocar 50 is representative of many types of surgical access devices which include a generally cylindrical seal housing 52 and an elongate cannula 54 that is adapted to extend across a body wall 56 into a body cavity 58. In the case of the depicted trocar 50, the cannula 54 is configured to extend through an abdominal wall 56 into a cavity, such as the abdominal cavity 58. The seal housing 52 includes a chamber 60 that is defined by an inner surface 62. The chamber 60 of the seal housing 52 communicates with a lumen 64 in the cannula 54 that is defined by an inner surface 66.

The trocar 50 is commonly used in laparoscopic surgery wherein the abdominal cavity 58 is pressurized with an insufflation gas in order to provide for organ separation and otherwise increase the size of the operative environment. With these features, the trocar 50 is adapted to receive an instrument 68 having an elongate configuration and an outer surface 70. The instrument 68 is illustrated to be a pair of scissors having a length sufficient to pass through the trocar 50 and into the cavity 58 to perform a surgical operation. Although scissors are illustrated in FIG. 1, it will be understood that the instrument 68 may include any variety of devices such as needles, retractors, scalpels, clamps, and various other surgical devices.

The seal housing 52 is configured to provide structural support for a seal mechanism 71, which includes an instrument seal or septum seal 72 and a zero closure seal or check valve seal 74. It is the function of the seals 72, 74 to prevent pressurized fluid or gas from escaping the cavity 58 whether the instrument 68 is operatively disposed in the trocar 50 or removed from the trocar. In either case, it is desirable that the valves 72, 74 be configured to produce minimal frictional forces as the instrument 68 is inserted into and removed from the trocar 50. Currently, the septum seal 72 will typically be formed of an elastomeric material so that an aperture 76 of the septum seal 72 is biased to seal against the outer surface 70 of the instrument 68. In order to avoid significant frictional forces, the aperture 76 of the septum seal 72 is preferably sized to a diameter slightly less than the diameter of the outer surface 70 of the instrument 68. However, since various instruments and various diameters for the outer surface 70 of the instrument 68 may be required in a particular surgery, the septum seal 72 may have to be changed in order to accommodate a range of instrument sizes.

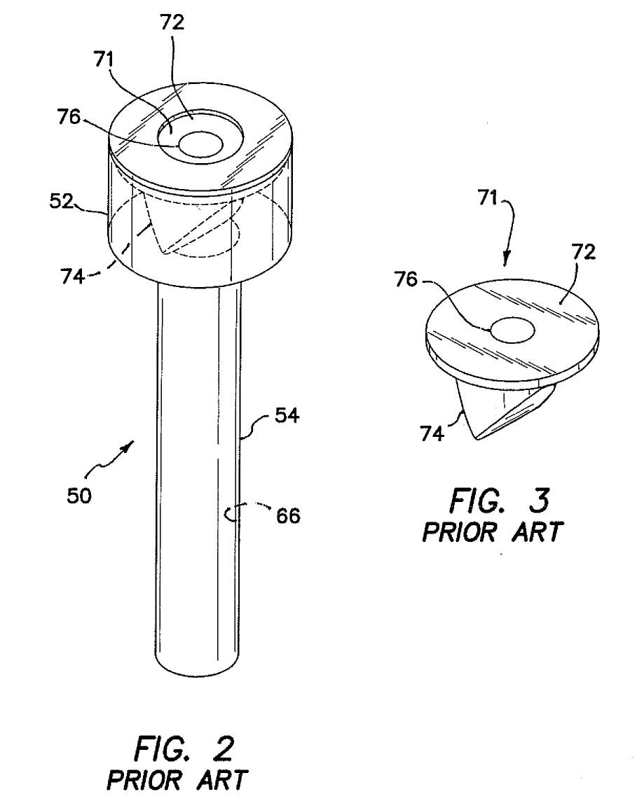

Referring to FIG. 3, the seal assembly 71 includes an instrument seal or septum seal 72. The septum seal 72 is generally sized and configured to provide a seal against a specific range of instrument diameters. The check valve 74 is not generally associated with a specific instrument size range since it provides a seal only when no instrument 68 is present within the passageway or lumen of the trocar 50 or catheter. Therefore, the check valves 74 associated with the prior art are generally substantially larger than the instruments 68 associated with the instrument size range associated with the septum seal 72.

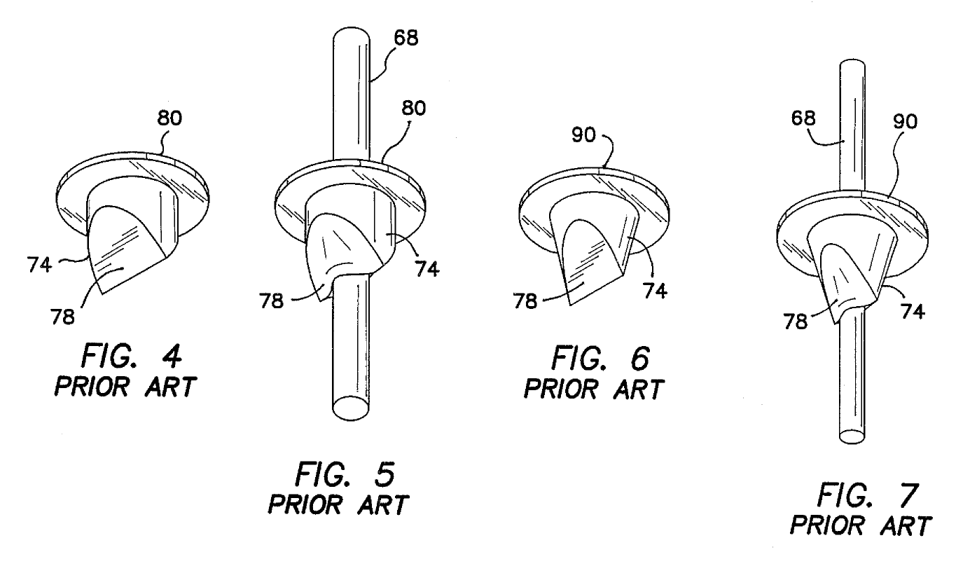

Referring to FIGS. 4-7, when an instrument 68 passes through the duckbill check valve 74 of the prior art, the seal provided by the check valve ceases to exist as gaps form between the instrument and the check valve. More particularly, the duckbill check valve 74 of the prior art has a binary arrangement where the check valve seal 78 is either in an open condition or a closed condition and has no sealing function when it is not in the closed condition. This binary arrangement exists in both cylindrical duckbill valves 80 (FIGS. 4 and 5) and in tapered, conical duckbill valves 90 (FIGS. 6 and 7).

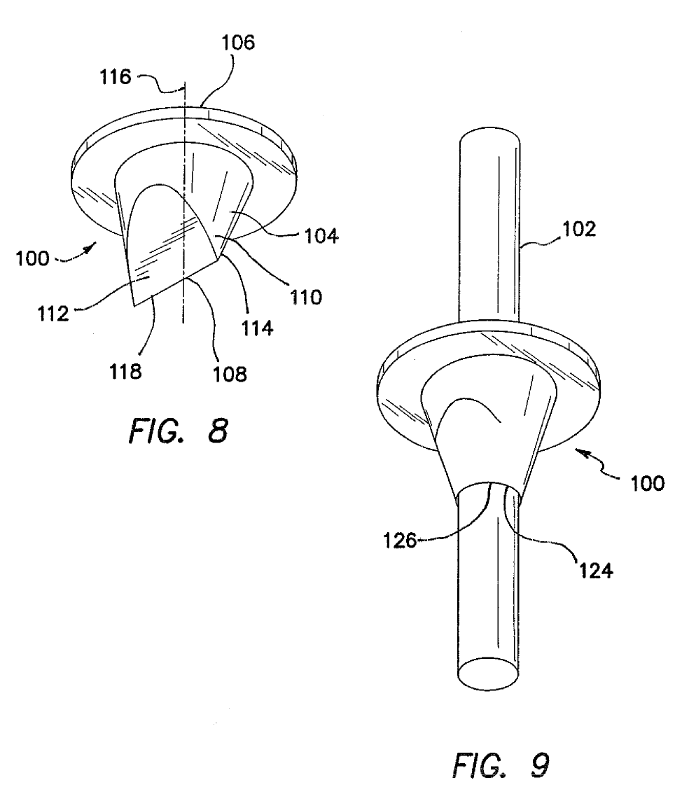

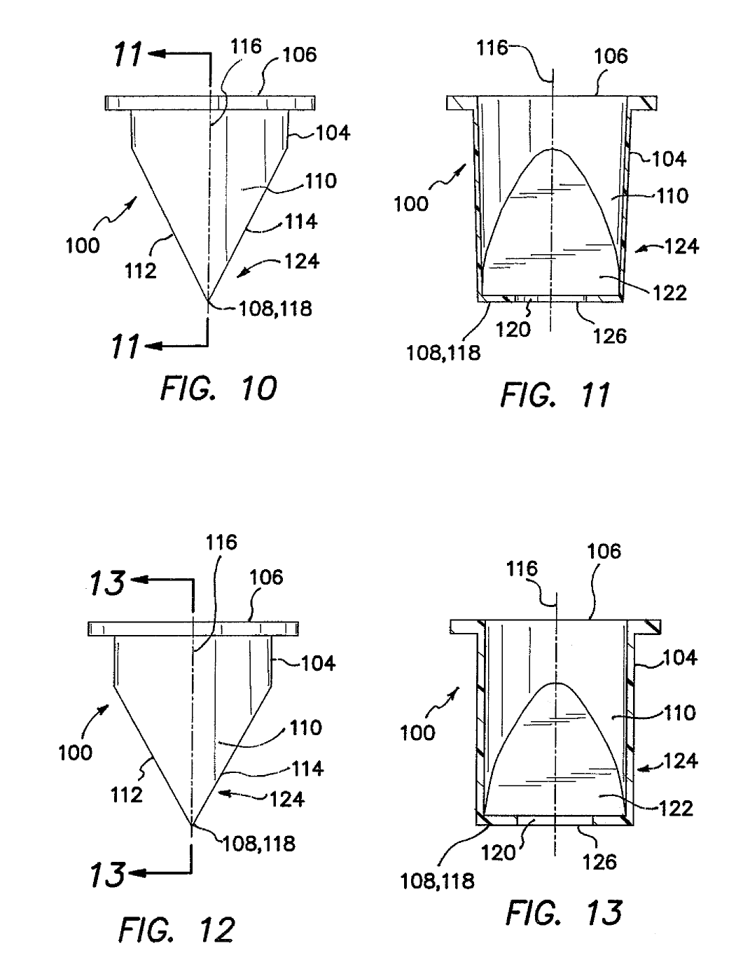

Referring to FIGS. 8-13, a duckbill check valve 100 for receiving an instrument 102 therethrough includes an elongate, hollow, elastomeric body 104 having a substantially circumferential shape. The elastomeric body 104 includes a first, proximal end 106 and a second, distal end 108. The proximal end 106 of the elastomeric body 104 is open and the distal end 108 of the elastomeric body is closed. In one aspect, the elastomeric body 104 may have a substantially conical shape (see FIGS. 8-11), while in another aspect, the elastomeric body may have a substantially cylindrical shape (see FIGS. 12-13). A distal portion 110 of the elastomeric body 104 includes a pair of opposed substantially flat portions 112, 114 extending distally at an angle toward a longitudinal axis 116 of the elastomeric body 104. The substantially flat portions 112, 114 intersect at the distal end 108 of the elastomeric body 104, thereby forming an edge 118 that is substantially perpendicular to the longitudinal axis 116 of the elastomeric body and may substantially intersect the longitudinal axis of the elastomeric body. A slit 120 extends along the length of the edge 118 at the distal end 108 of the elastomeric body 104 and extends from an outer surface of the elastomeric body to an inner surface of the elastomeric body. The periphery of the slit 120 is no larger than the periphery of the instrument 102 that is to be received by the duckbill check valve 100. In one aspect, the periphery of the slit 120 is smaller than the periphery of the instrument 102 that is inserted therethrough. In one aspect, the duckbill check valve 100 is formed of an elastomeric material, such as a low durometer polymer. The slit 120 and the substantially flat portions 112, 114 form an elastomeric seal 124.

In other aspects, the duckbill check valve may be a multiple duckbill valve, such as a double duckbill valve or triple duckbill valve, with a slit at the distal end of each duckbill portion. In such embodiments, the periphery of the combined slits is less than the periphery of an instrument to be inserted therethrough.

With the periphery of the slit 120 being smaller than the periphery of an instrument 102 being received by the check valve 100, the slit in the duckbill elastomeric seal 124 must stretch to fit around the periphery of the instrument traversing the passageway of the check valve, thereby forming a seal around the instrument. The distal-most portion of the duckbill check valve 100, including the slit 120, may be sized to allow passage of a specific range of instrument diameters. In this manner, the periphery of the slit 120 is smaller than the periphery of the smallest instrument within the range of instruments. The stretched distal seal portion returns to a slit configuration and again forms a duckbill check valve upon removal of the instrument from the passageway. The substantially flat portions 112, 114 are sized and configured to seal under retrograde pressure such that the slit 120 provides a zero seal in the absence of an instrument positioned within the slit and provides an instrument seal in the presence of an instrument positioned within the slit.

The flat portions 112, 114 may be more elastic than the remainder of the elastomeric body 104. In this manner, the flat portions 112, 114 include elastomeric properties allowing the check valve to form a seal around an instrument inserted therein while the remainder of the elastomeric body 104 provides support for the flat portions. Means for providing varying elastomeric properties include manufacturing the flat portions 112, 114 to be thinner than the remainder of the elastomeric body 104, making the flat portions of a material having a lower durometer than the remainder of the elastomeric body, and other means that are well known in the art.

It can be appreciated that having a single seal member instead of multiple seal members decreases frictional resistance. In addition to reducing friction, the present invention provides a more durable seal member since inserted instruments approach the potential orifice or septum at a substantially greater angle than would be the case with a flat or conical septum. Therefore, sharp or pointed instruments are less likely to cut, tear or pierce the seal of the present invention.

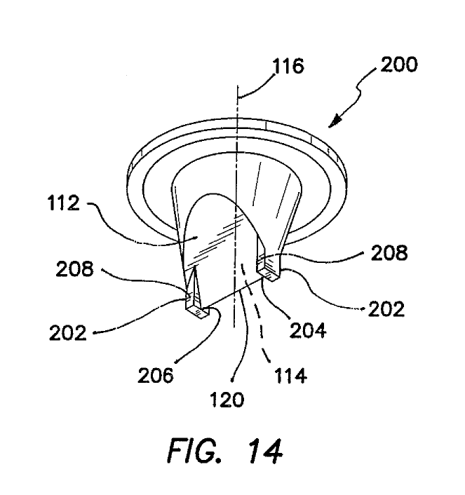

Referring to FIG. 14, in one aspect a duckbill check valve 200 may include one or more reinforcing structural members 202. In one aspect, the duckbill check valve 200 includes a reinforcing structural member 202 at each end 204, 206 of the slit 120. Each of the reinforcing structural members 202 includes a protuberance 208 projecting away from each of the flat portions 112, 114 of the check valve, in a direction generally perpendicular to a plane formed by the longitudinal axis 116 and the distal edge 118 of the check valve, and extending proximally into each of the flat portions of the check valve. The reinforcing structural members 202 resist tearing or elongation of the slit 120 when under the influence of an inserted instrument 102. The reinforcing structural members 202 are sized and configured to assist in returning the opened seal to a closed condition when an instrument 102 is removed from the passageway.

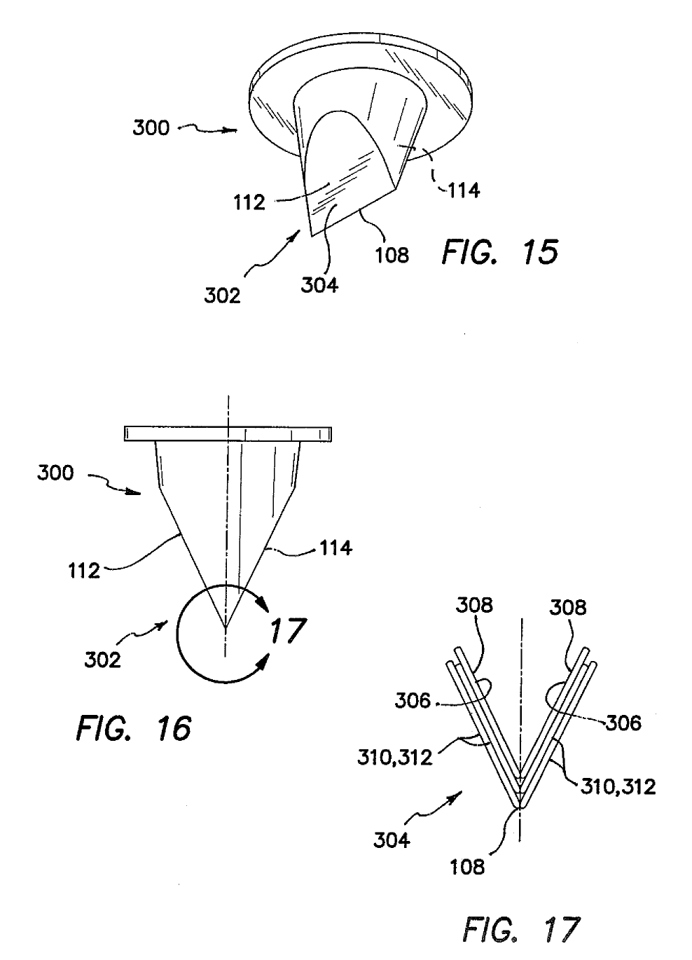

Referring to FIG. 15, a duckbill check valve 300 may include a sealing portion 302, such as the substantially flat portions 112, 114, having a plurality of fine bristles 304. The bristles 304 may be arranged to oppose near the distal end 108 of the check valve 300 to form a fluid-tight seal under the influence of retrograde pressure. The bristles 304 may include a substantially non-elastic material for the sealing portions. Substantially non-elastic materials tend to have a lower coefficient of friction than elastomeric materials, but they are not generally suited for fluid-tight seals.

Referring to FIGS. 16 and 17, the bristles 304 may be arranged in several layers that provide specific characteristics for the seal. In one embodiment, the seal includes an innermost layer 306 of fine, rigid, uncoated bristles 308 that provide a nearly friction-free pathway against an instrument 102 inserted therethrough and a second, outer layer 310 of fine, rigid bristles 312 that are coated with an elastomeric sealing material that provides a seal as the second layer of bristles interacts with the first layer of bristles. Additional outer layers 310 of coated bristles 312 may be added to further improve the sealing characteristics.

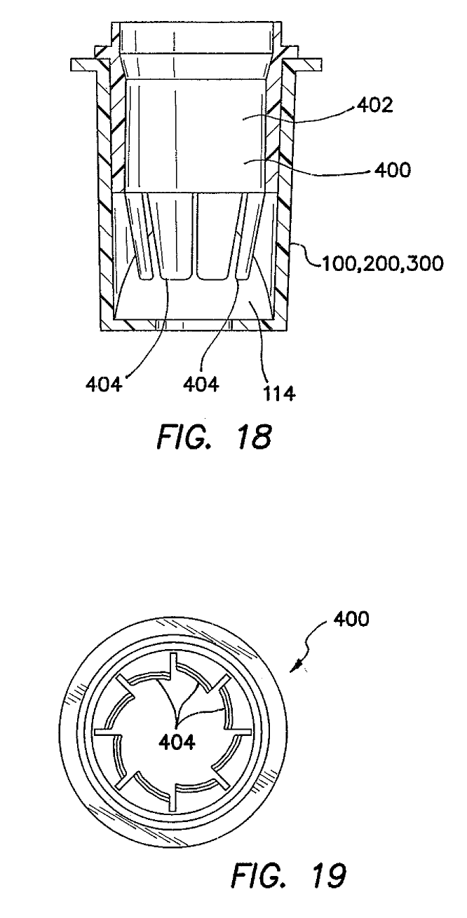

Referring to FIGS. 18 and 19, a shield 400 may be used with the check valve 100, 200, 300 to protect the check valve during the insertion and removal of surgical instruments. The shield 400 includes a tubular member 402 having a proximal end and a distal end. A plurality of blades or leaflets 404 protrude from the distal end of the tubular member 402. The shield 400 is placed inside the check valve such that the blades or leaflets 404 cover the inner surface of the flat portions 112, 114 of the check valve 100, 200, 300. In one embodiment, adjacent blades or leaflets 404 may overlap one another. The blades or leaflets 404 are located within the region of the flat portions 112, 114 of the check valve. By so locating the shield 400, the drag force required to insert or remove instruments may be reduced by allowing the instruments to slide on the lubricious material of the shield rather than on the soft, flexible material of the check valve 100, 200, 300. The shield 400 can also be used to support the check valve 100, 200, 300 and to restrict check valve movement in the axial direction of the check valve and surgical instruments. The shield 400 may be formed of a rigid polymeric material, such as a plastic, and may operate to center and guide an instrument as it is inserted through the check valve 100, 200, 300.

Many alterations and modifications may be made by those having ordinary skill in the art without departing from the spirit and scope of the invention. For example, it is contemplated that the geometry, material, and placement of the check valve may be modified for different applications. Therefore, it must be understood that the illustrated embodiments have been set forth only for the purposes of examples and that they should not be taken as limiting the invention.

1. A duckbill check valve for receiving an instrument therethrough, comprising:
a substantially circumferential hollow elastomeric body having a first, proximal end that is open and a second, distal end that is closed;
a pair of opposed substantially flat portions at an angle to a longitudinal axis of the elastomeric body, the substantially flat portions extending distally toward one another and intersecting at the distal end of the elastomeric body, thereby forming an edge, the edge being substantially perpendicular to the longitudinal axis of the elastomeric body; and
a slit along the edge at the distal end of the body, the periphery of the slit being no larger than the periphery of the instrument to be received by the check valve, the slit extending from an outer surface of the elastomeric body to an inner surface of the elastomeric body,
wherein the slit provides a zero seal in the absence of an instrument positioned within the slit and an instrument seal in the presence of an instrument positioned within the slit.

2. The check valve of claim 1, the elastomeric body having a substantially cylindrical shape.

3. The check valve of claim 1, the elastomeric body having a substantially conical shape.

4. The check valve of claim 1, the periphery of the slit being smaller than the periphery of the instrument to be received by the check valve.

5. The check valve of claim 1, further comprising:
a reinforcing structural member at each end of the slit, each of the structural members comprising a protuberance projecting away from each of the flat portions of the check valve generally perpendicular to a plane formed by the longitudinal axis and the distal edge of the check valve and extending proximally into each of the flat portions of the check valve.

6. The check valve of claim 1, wherein the check valve is formed of an elastomeric material including a low durometer polymer.

7. The check valve of claim 1, wherein the substantially flat portions are more elastic than the remainder of the elastomeric body.

8. The check valve of claim 7, wherein the substantially flat portions have a thickness smaller than the thickness of the remainder of the elastomeric body.

9. The check valve of claim 7, wherein the substantially flat portions have a lower durometer than the remainder of the elastomeric body.

10. The check valve of claim 1, further comprising:
a shield including,
a tubular member having a proximal end and a distal end, and
a plurality of blades protruding distally from the distal end of the tubular member;
the shield being placed inside the check valve such that the blades engage the inner surface of the flat portions of the check valve.

11. The check valve of claim 10, the shield being formed from a rigid plastic material.

12. The check valve of claim 10, wherein adjacent blades overlap one another.

13. The check valve of claim 10, the shield operating to center and guide the surgical instrument through the blades before expanding the slit of the check valve.

14. A duckbill check valve for receiving an instrument therethrough, comprising:
a substantially circumferential hollow elastomeric body having a first, proximal end that is open and a second, distal end that is closed;
a pair of opposed substantially flat portions at an angle to a longitudinal axis of the elastomeric body, the substantially flat portions extending distally toward one another and intersecting at the distal end of the elastomeric body, thereby forming an edge, the edge being substantially perpendicular to the longitudinal axis of the elastomeric body;
a slit along the edge at the distal end of the body, the periphery of the slit being smaller than the periphery of the instrument to be received by the check valve, the slit extending from an outer surface of the elastomeric body to an inner surface of the elastomeric body; and
a shield including a tubular member having a proximal end and a distal end, and a plurality of blades protruding distally from the distal end of the tubular member, the shield being placed inside the check valve such that the blades engage the inner surface of the flat portions of the check valve,
wherein the slit provides a zero seal in the absence of an instrument positioned within the slit and an instrument seal in the presence of an instrument positioned within the slit,
the check valve being formed of an elastomeric material including a low durometer polymer,
the shield being formed from a rigid plastic material, and
the shield operating to center and guide the surgical instrument through the blades before expanding the slit of the check valve.

15. The check valve of claim 14, the elastomeric body having a substantially cylindrical shape.

16. The check valve of claim 14, the elastomeric body having a substantially conical shape.

17. The check valve of claim 14, further comprising;
a reinforcing structural member at each end of the slit, each of the structural members comprising a protuberance projecting away from each of the flat portions of the check valve generally perpendicular to a plane formed by the longitudinal axis and the distal edge of the check valve and extending proximally into each of the flat portions of the check valve.

18. The check valve of claim 14, wherein the substantially flat portions are more elastic than the remainder of the elastomeric body.

19. The check valve of claim 18, wherein the substantially flat portions have a thickness smaller than the thickness of the remainder of the elastomeric body.

20. The check valve of claim 18, wherein the substantially flat portions have a lower durometer than the remainder of the elastomeric body.

21. The check valve of claim 14, wherein adjacent blades overlap one another.

22. A duckbill check valve for receiving an instrument therethrough, comprising:
a substantially circumferential hollow elastomeric body having a first, proximal end that is open and a second, distal end that is closed; and
a pair of opposed substantially flat portions at an angle to a longitudinal axis of the elastomeric body, the substantially flat portions extending distally toward one another and intersecting at the distal end of the elastomeric body, thereby forming an edge, the edge being substantially perpendicular to the longitudinal axis of the elastomeric body, the flat portions comprising a plurality of bristles arranged in opposition,
wherein the bristles provide a zero seal in the absence of an instrument passing therethrough and an instrument seal in the presence of an instrument passing therethrough.

23. The check valve of claim 22, the elastomeric body having a substantially cylindrical shape.

24. The check valve of claim 22, the elastomeric body having a substantially conical shape.

25. The check valve of claim 22, wherein the elastomeric body is formed of an elastomeric material including a low durometer polymer.

26. The check valve of claim 22, the bristles including a substantially non-elastic material.

27. The check valve of claim 22, the plurality of bristles comprising:
a first, innermost layer of uncoated bristles; and
at least one outer layer of bristles coated with an elastomeric material.

28. The check valve of claim 22, further comprising:
a shield including,
tubular member having a proximal end and a distal end, and
a plurality of blades protruding distally from the distal end of the tubular member;
the shield being placed inside the check valve such that the blades engage the inner surface of the flat portions of the check valve.

29. The check valve of claim 28, the shield being formed from a rigid plastic material.

30. The check valve of claim 28, wherein adjacent blades overlap one another.

31. The check valve of claim 28, the shield operating to center and guide the surgical instrument through the blades before separating the bristles.

32. A duckbill check valve for receiving an instrument therethrough, comprising:
a substantially circumferential hollow elastomeric body having a first, proximal end that is open and a second, distal end that is closed;
a pair of opposed substantially flat portions at an angle to a longitudinal axis of the elastomeric body, the substantially flat portions extending distally toward one another and intersecting at the distal end of the elastomeric body, thereby forming an edge, the edge being substantially perpendicular to the longitudinal axis of the elastomeric body, the flat portions comprising a plurality of bristles arranged in opposition; and
a shield including a tubular member having a proximal end and a distal end, and a plurality of blades protruding distally from the distal end of the tubular member, the shield being placed inside the check valve such that the blades engage the inner surface of the flat portions of the check valve,
wherein the bristles provide a zero seal in the absence of an instrument passing therethrough and an instrument seal in the presence of an instrument passing therethrough,
the elastomeric body being formed of an elastomeric material including a low durometer polymer,
the bristles including a substantially non-elastic material; the shield being formed from a rigid plastic material, and
the shield operating to center and guide the surgical instrument through the blades before separating the bristles.

33. The check valve of claim 32, the elastomeric body having a substantially cylindrical shape.

34. The check valve of claim 32, the elastomeric body having a substantially conical shape.

35. The check valve of claim 32, the plurality of bristles comprising:
a first, innermost layer of uncoated bristles; and
at least one outer layer of bristles coated with an elastomeric material.

36. The check valve of claim 32, wherein adjacent blades overlap one another.
